(19) United States
(12) Patent Application Publication
(10) Pub. No.: US 2005/0209345 A1
(43) Pub. Date: Sep. 22, 2005

(54) Lymphatic drug delivery system

(76) Inventor: William N. Charman, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/480,961

(22) PCT Filed: Jun. 14, 2002
(86) PCT No.: PCT/AU02/00779

§ 371 Date: Apr. 22, 2005

(30) Foreign Application Priority Data
Jun. 15, 2001 (AU) PR 5730

(51) Int. Cl. A61K 47/00; A61K 9/00

(52) U.S. Cl. 514/784; 424/400

(57) ABSTRACT

This invention relates to methods of delivering a medium to highly lipophilic drug to the systemic circulation through the lymphatic transport system including the oral administration to a subject in the fasted state of a formulation comprising the lipophilic drug and a component capable of stimulating the production of endogenous lipid, or a component selected from medium to long chain lipids, pharmaceutically acceptable surfactants and combinations thereof, wherein the component is present in an amount sufficient to enhance or promote lymphatic transport of the lipophilic drug, and corresponding methods for avoiding lymphatic transport.

### BACKGROUND OF THE INVENTION

1. Field of the Invention

This invention relates to the oral delivery of drugs via the lymphatic transport system, more particularly to the delivery of medium to highly lipophilic drugs to the systemic circulation via the lymphatic transport system. The invention provides methods for the oral delivery of medium to highly lipophilic drugs, methods of stimulating production/translocation of endogenous lipid to promote lymphatic transport, methods of avoiding the requirement to administer medium to highly lipophilic drugs in the fed state, methods of avoiding prolongation of the QTc interval (of an ECG) following administration of a medium to highly lipophilic drug and methods of delivering medium to highly lipophilic drugs at high concentrations in the lymph, or, in the case of medium to highly lipophilic cardiac active drugs, at high concentrations to the systemic circulation near the heart.

2. Disclosure of Related Art

The advent of combinatorial chemistry and high throughput screening has resulted in the rapid identification of many highly potent new chemical entities. Coincident with the increasing use of these technologies, however, has been a developing trend towards the identification of lead compounds with higher molecular weights and log octanol/water partition coefficients (Log P), and lower water solubilities. Whilst these attributes conspire to provide high affinity binding to drug targets, they do not necessarily engender ideal biopharmaceutical provides. Indeed, historically, many of these compounds have failed to progress into clinical development, due to a reticence to progress drugs having less than ideal pharmacokinetic/absorptive profiles.

Highly lipophilic drugs, as well as medium lipophilic drugs, provide both challenges, and potentially, some specific advantages to a pharmaceutical scientist. Thus, their poor water solubility almost inevitably leads to low oral bioavailability from the conventional dose forms. However, formulation strategies for lipophilic poorly water soluble compounds have advanced significantly in recent years and the use of lipid based emulsions and microemulsions, and self-emulsifying and self-microemulsifying formulations has led to the effective development of many such compounds with acceptable oral bioavailability. The ability to efficiently deliver highly lipophilic drug molecules, especially in combination with lipid based delivery systems has also led to a renewed interest in intestinal lymphatic drug transport.

After absorption into the enterocycte, the vast majority of orally administered drugs rapidly diffuse across the cell, are absorbed into the capillaries of the portal vein and are thereby processed via the liver prior to gaining access to the systemic circulation. Highly lipophilic drug molecules, however, may associate with lymph lipoproteins in the enterocyte and gain access to the mesenteric (intestinal) lymphatics, effectively by-passing the liver and gaining access to the systemic circulation via the thoracic lymph duct.

The extremely high drug concentrations attainable in the lymph (up to 1000×higher than plasma concentrations) offer drug delivery advantages in addition to reduced first pass metabolism for lymphatically transported drugs, including, specific delivery to lymph resident B and T lymphocytes and the opportunity to target a principle pathway for the dissemination of tumour metastases. Accordingly lymph transport of drugs useful in interferon and immunomodulator therapy, and cytotoxic agents may present particular advantages. Suggestions of a major role for the lymph and lymphoid tissue in the development of HIV infection (see for example G. Pantaleo, C. Graziosi, J. F. Demarest, O. J. Cohen, M. Vaccarezza, Gantt, K, C. Muro-Cacho, A. S. Fauci, Role of Lymphoid organs in the pathogenesis of human immunodeficiency virus (HIV) infection. Immunol. Rev. 140 (1994) 105-130 and G. Pantalco, C. Graziosi, A. S. Fauci, The role of lymphoid organs in the immunopathogenesis of HIV infection. Aids 7 (1993) S19-23) has also heightened interest in the lymph as an anti-viral target in AIDS patients.

Specificity for access to the intestinal lymph (as opposed to the portal blood) is provided by the differences in endothelial architecture between lymph and blood vessels. Whereas the vascular endothelial barrier features tight inter-endothelial junctions and a continuous basal lamina, the lymphatic vessels are characterised by either discontinuous or absent basal lamina and relatively wide inter-endothelial junctional distances (see for example N. S. Kumar, C. M. Mansbach, Prechylomicron transport vesicle: isolation and partial characterization. Am. J. Physiol. 276 (1999) G378-86). The transcellular access of small, lipophilic molecules into either the lymph or the blood is therefore relatively unimpeded and under these circumstances absorption into the blood predominates, due to significantly higher blood flow compared to lymph flow aiding the mass transport process. However, for either large (>10,000 Da) or extremely hydrophilic compounds or large colloidal structures, transcellular transport into the blood is significantly hindered, and paracellular access across the more “leaky” lymphatic endothelium is favoured.

Lymphatic transport of dietary and formulation derived lipids is assured by the assembly of the products of triglyceride (TG) resynthesis into colloidal structures (lipoproteins), the size of which precludes absorption into the blood. In the case of a co-administered drug, therefore, appreciable lymphatic transport of small molecules will only occur when the drug molecules favourably associate with lymph lipoproteins, and do so with an avidity sufficient to overcome the significantly higher mass transfer of fluid through the portal blood.

Charman and co-workers have previously suggested that a log partition coefficient (log P) value in excess of about 5 and a TG solubility in excess of about 50 mg/mL is a minimum requirement for appreciable lymphatic transport (see for example W. N. Charman, V. J. Stella, Estimating the maximal potential for intestinal lymphatic transport of lipophilic drug molecules, Int. J. Pharmaceut 34 (1986) 175-178). This estimation was based on the difference between portal blood and lymph flow (500:1) and the realisation that the actual lipid load in the lymph accounted for approximately 1% of the total lymph flow, and therefore that the mass transfer ratio between lymph lipid and portal blood was of the order of 1 to 50,000. Since lymphatically transported drugs are transported in association with the TG core of chylomicrons it was suggested that for a drug molecule to be preferentially transported into the lymph, it should have at least 50,000 times more affinity for the lymph lipid when compared with portal blood (i.e. log P>4.7).

In contrast to these general indicators of lymphatic transport (log P>5, lipid solubility>50 mg/g) Nankervis and co-workers have recently demonstrated an inverse relationship between lipid solubility and lymphatic uptake for three highly lipophilic retinoids (temarotene, etretnate and isotretinoin) after oral absorption. The authors suggested that the extent of lymphatic transport of the retinoids from the vehicles in which they were most soluble was limited by their preference for partition into the co-administered lipid vehicle rather than partition into the enterocycte (see for example R. Nankervis, S. S. Davis, N. H. Day, P. N. Shaw, Intestinal lymphatic transport of three retinoids in the rat after oral administrations: effect of lipophilicity and lipid vehicle. Int. J. Pharmaceut. 130 (1996) 57-64.

These observations highlight the complexities associated with the processing of a lipophilic drug prior to intestinal lymphatic drug transport. Lymphatically transported lipophilic drugs associate with lipidic microdomains in the intestinal lumen (presumably driven by simple partitioning behaviour) and also are found to associate very specifically with lymph lipoproteins. However, the mechanism of association of lipophilic drugs with lymph lipoproteins is not well described. Indeed, drug-lipoprotein association has been historically ascribed simply to partition phenomena and little is known as to the site of association, the contributions of microdistribution patterns within the enterocycte and the potential role of binding and transfer proteins.

The apparent requirement for drug association with intestinal derived lipoproteins as a prerequisite for drug transport via the intestinal lymphatics has led to the convention of administering highly lipophilic drugs with food or at meal times. This has also led to the common use of lipids and lipid based formulations to enhance intestinal lymphatic transport (see for example W. N. Charman, C. J. H. Porter, Lipophilic prodrugs designed for intestinal lymphatic transport. Adv. Drug. Deliv. Rev. 19 (1996) 149-169, C. J. H. Porter, Drug delivery to the lymphatic system. Criti. Rev. Ther. Drug Carrier Syst. 14 (1997) 333-93 and S. Muranishi, Drug targeting towards the lymphatics. Adv. Drug. Res. 21 (1991) 1-38). The basic premise underlying the design of the formulations is that the digestion and absorption of co-administered lipids stimulates lipid turnover through the enterocyte, enhancing chylomicron synthesis and thereby increasing the lipoprotein-based lipid sink into which drugs may partition.

Lipid based formulations may also enhance drug absorption generically via improvements in dissolution and solubilisation within the intestinal milieu, a reduction in gastric emptying rate, and increases in mucosal permeability and thereto, have the potential to both enhance the overall extent of absorption as well as increase the proportion of what is absorbed, being transported to the systemic circulation via the intestinal lymph (see for example A. J. Humberstone, W. N. Charman, Lipid based vehicles for the oral delivery of poorly water soluble drugs. Adv. Drug. Deliv. Rev. 25 (1997) 103-128 and W. N. Charman, C. J. Porter, S. Mithani, J. B. Dressman, Physiochemical and physiological mechanisms for the effects of food on drug absorption: the role of lipids and pH. J. Pharm. Sci. 86 (1997) 269-82).

Many of the most recent reports detailing examples of lymphatic drug transport, have originated from development groups within the industry where the primary goal of the project has been an enhancement in overall bioavailability, with a parallel investigation of the extent of lymphatic transport. Consequently, the majority of these studies have examined the extent of lymphatic drug transport after oral administration in relatively complex and generally highly dispersed lipid vehicles which makes assessment of the intrinsic lymph directing capacity of individual components more difficult. These studies have also been almost exclusively performed using rats where relatively large volumes of lipid (relative to body weight) must be administered.

For example, Hauss and co-workers examined the lymphatic transport in rats of a lipophilic, poorly water-soluble, anti-inflammatory agent and potent inhibitor of leukotriene (LTB₄) synthesis, ontazolast, formulated as an aqueous based suspension and four different lipid based emulsion formulations and determined the effects of formulation on lymphatic transport and systemic blood absorption (see for example D. J. Hauss, S. E. Fogal, J. V. Ficorilli, C. A. Price, T. Roy, A. A. Jayaraj, J. J. Keirns, Lipid-based delivery systems for improving the bioavailability and lymphatic transport of a poorly water-soluble LTB4 inhibitor. J. Pharm. Scie. 87 (1998) 164-9). The formulations used included a simple solution of ontazolast in Peceol (a mixture of mono and diglycerides of oleic acid), an oil (soybean oil) in water emulsion and two self emulsifying drug delivery systems (SEDDS) comprising mixtures of Gelucire 44/14 (containing mono-, di- and triglycerides, PEG 1500 mono- and diesters of fatty acids and free PEG 1500) and Peceol. Whilst the complexity of the lipid based formulation made comparison of individual lipid effects difficult, the total amount of lymphatically transported ontazolast over the 24 h post dosing period was found to be approximately 20 to 25-fold greater from the lipid based SEDDS systems and 50-fold greater from the soybean emulsion, when compared with the aqueous based suspension formulation. The authors reported a concurrent increase in the lymphatic transport of triglyceride lipid that mirrored the trend in lymphatic drug transport.

Hause et al. also studied the ability of a lipid-based formulation to enhance the intestinal lymphatic uptake and systemic absorption of a lipophilic lipid regulator Cl-917 after administration as either a lipid-free suspension formulation or a lipid-based emulsion in conscious rats (see for example D. J. Haus, S. Mehta, G. W. Radebaugh, Targeted lymphatic transport and modified systemic distribution of Cl-976, a lipophilic lipid-regulator drug, via a formulation approach. Int. J. Pharmaceut. 108 (1994) 85-93). The total amount of Cl-976 transported in lymph over 14 h, as a percent of the administered dose was 7 times greater for the lipid-based emulsion when compared with the suspension formulation. Although the absolute amount of drug transported lymphatically was low (<1%), the major proportion of systemic availability was suggested to be due to lymphatic transport via drug association with chylomicrons.

Kwei and co-workers studied the lymphatic transport of a poorly water soluble (<I μg/mL) and highly lipid soluble (>80 mg/mL in soybean oil), 5 a reductase enzyme inhibitor, MK-386, from three oral formulations: an aqueous suspension, a mixed lipid/surfactant (Imiwitor®, mono and diglycerides of capric and caprylic acids/polysorbate 80) formulation and a long chain lipid (soybean oil) solution formulation after oral administration of radiolabelled MK-386 to rates (see for example G. Y. Kwei, L. B. Novak, L. H. Hettrick. E. R. Reiss, E. K. Fong, T. V. Olah, A. E. Loper, Lymphatic uptake of MK-386, a sterol 5-alpha reductase inhibitor, from aqueous and lipid formulations. Int. J. Pharmaceut. 164 (1998) 37-44). Interestingly, the rank order of the total amount of MK-386 transported into the mesenteric lymph over a 6 h post-dosing period was found to be aqueous suspension>mixed lipid/surfactant system>soybean oil, i.e. greatest for the lipid-free formulation. These studies were complicated by the fact that the 6 h sampling time employed was not sufficient to cover the complete absorption profile. Furthermore, the mixed lipid/surfactant formulation contained a relatively large amount of surfactant, notably in excess of the critical micelle concentration and this may have affected the rate and extent of dissolution and subsequently the absorption profile of the poorly water soluble MK-386. Nevertheless, MK-386 was primarily transported to the systemic circulation by the intestinal lymph (after administration of all three formulations), with up to 80-fold higher levels of radioactivity seen in the lymph compared with the portal blood.

A number of other studies have demonstrated that significant lymphatic transport may occur in the absence of co-administered lipid. For example, Nishigaki and co-workers reported a two-fold increase in lymphatic transport of retinyl palmitate after administration in an aqueous polysorbate 80 micellar solution compared with a lipid solution formulation (see for example R. Nishigaki, S. Awazu, M. Hanano, T. Fuwa, The effect of dosage form on absorption of vitamin A into lymph. Chem. Pharm. Bull. 24 (1976) 3207-3211) and the lymphatic absorption of cyclosporin was found to be significantly greater (5-10 fold) after administration in a simple micellar solution formulation when compared with a lipid solution or mixed micellar formulations (see for example K. Takada, H. Yoshimura, H. Yoshikawa, S. Muranishi, T. Yasamura, S. Oka, Enhanced lymphatic delivery of cyclosporin A by solubilisers and intensified immunosuppressive activity against mice skin allograft. Pharm. Res. 3 (1986) 48-51). Similarly, no significant differences were reported in the extent of lymphatic transport of mepitiostane after administration in a polysorbate 80 micellar solution or a triglyceride lipid vehicle (see for example T. Ichihashi, H. Kinoshita, Y. Takagishi, H. Yamada, Effect of oily vehicles on absorption of mepitiostane by the lymphatic system in rats. J. Pharm. Pharmacol. 44 (1992) 560-4). In these examples, the relatively high extent of lymphatic transport observed after oral administration in lipid-free formulations may be a function of (1) enhanced overall drug absorption occurring as a result of efficient solubilisation in the (usually) surfactant rich formulations, (2) enhanced drug absorption facilitated by the high surfactant concentration leading to a generalised increase in membrane permeability and (3) partitioning of drugs into lymph lipoproteins synthesised from endogenous lipids or the products of surfactant hydrolysis. Notwithstanding these latter few examples, however, in the majority of cases, lipid based formulations appear to enhance the lymphatic transport of co-administered highly lipophilic drugs in rats.

Although these rat studies indicate that lipid based formulations may lead to enhanced lymphatic transport in the rat, it is difficult to extrapolate such date to higher species as the largely constant bile flow in a rat precludes attainment of representative pre- and post-prandial states, and formulations administered to rats are often not relevant to higher species.

### SUMMARY OF THE INVENTION

It has now been surprisingly found that the co-administration with a medium to highly lipophilic drug of a medium to long chain lipid, pharmaceutically acceptable surfactant or combination thereof, or of a component capable of stimulating endogenous lipid production or translocation, can promote transport of the lipophilic drug into the lymphatic transport system.

Accordingly, in a first aspect, the invention provides a method of delivering a medium to highly lipophilic drug to the systemic circulation through the lymphatic transport system comprising the oral administration to a subject in the fasted state of a formulation comprising said lipophilic drug and a component selected from medium to long chain lipids, pharmaceutically acceptable surfactants and combinations thereof, wherein said component is present in an amount sufficient to enhance or promote lymphatic transport of said lipophilic drug.

According to a second aspect, there is provided a method of delivering a medium to highly lipophilic drug to the systemic circulation through the lymphatic transport system comprising the oral administration to a subject in a fasted state of a formulation comprising said lipophilic drug and a component capable of stimulating sufficient production of endogenous lipid to allow transport of the drug into the lymphatic transport system.

According to a third aspect the invention provides a method of stimulating the production of endogenous lipid in a subject in a fasted state in sufficient quantity to enhance or promote transport of a medium to highly lipophilic drug into the lymphatic transport system for delivery to the systemic circulation, said method comprising orally administering to said subject in combination with said lipophilic drug an effective amount of a component selected from medium to long chain lipids, pharmaceutically acceptable surfactants and combinations thereof, whereby the amount of lipid transported to said lymphatic transport system following administration of said medium to long chain lipid drug is greater than the amount of lipid administered with said lipophilic drug.

According to a fourth aspect there is provided a method of avoiding the requirement of administering a medium to highly lipophilic drug in the fed state, said method comprising formulating said lipophilic drug with a component selected from medium to long chain lipids, pharmaceutically acceptable surfactants and combinations thereof, such that oral administration of the formulation in the fed or fasted state allows delivery of the drug to the systemic circulation through the lymphatic transport system.

In a fifth aspect there is provided a method of avoiding the requirement of administering a medium to highly lipophilic drug in the fed state, said method comprising formulating said lipophilic drug with a component capable of stimulating the production of endogenous lipid, such that oral administration of the formulation in the fed or fasted state allows delivery of the lipophilic drug to the systemic circulation through the lymphatic transport system.

In a sixth aspect there is provided a method of avoiding prolongation of the QT_{c} interval of an electrocardiogram (ECG) following oral administration of a medium to highly lipophilic drug, said method comprising the formulation of said lipophilic drug in the absence of medium to long chain lipids, or other component capable of stimulating the production of endogenous lipid, and the administration of said formulation in the fasted state.

In a seventh aspect the present invention provides a method of delivering a medium to highly lipophilic drug in high concentration to the lymph comprising the oral administration to a subject in the fasted state of a formulation comprising said lipophilic drug and a component selected from medium to long chain lipids, pharmaceutically acceptable surfactants and combinations thereof, wherein said component is present in an amount sufficient to enhance or promote lymphatic transport of said lipophilic drug.

In a eighth aspect the present invention provides a method of delivering a medium to highly lipophilic drug in high concentration to the lymph comprising the oral administration to a subject in the fasted state of a formulation comprising said lipophilic drug and a component capable of stimulating sufficient endogenous lipid production to allow transport of the drug to the lymphatic transport system in high concentration.

According to a ninth aspect the invention provides a method of targeting delivery of a medium to highly lipophilic drug to the heart comprising the oral administration to a subject in the fasted state of a formulation comprising said lipophilic drug and a component selected from medium to long chain lipids, pharmaceutically acceptable surfactants and combinations thereof, wherein said lipid is present in an amount sufficient to enhance or promote lymphatic transport of said lipophilic drug.

According to a tenth aspect there is provided a method of targeting delivery of a medium to highly lipophilic drug to the heart comprising the oral administration to a subject in a fasted state of a formulation comprising said drug and a component capable of stimulating sufficient production of endogenous lipid to allow transport of the drug into the lymphatic transport system.

According to an eleventh aspect the present invention provides a pharmaceutical formulation comprising a highly lipophilic drug and a component selected from medium to long chain lipids, pharmaceutically acceptable surfactants and combinations thereof, wherein said component is present in an amount sufficient to enhance or promote lymphatic transport of said lipophilic drug when administered in the fasted state.

In a twelfth aspect the present invention provides a pharmaceutical formulation comprising a highly lipophilic drug and a component capable of stimulating sufficient production of endogenous lipid following administration to allow transport of the drug into the lymphatic transport system.

### DESCRIPTION OF PREFERRED EMBODIMENTS

The present invention can provide a number of advantages over conventional methods for the delivery of medium to highly lipophilic drugs to their sites of action within the subject. For example, in the case of drugs useful in the treatment of heart related conditions, it is possible to deliver the drug in high concentrations to the systemic circulation at a position in the systemic circulation close to the heart. This is possible because the lymphatic transport system meets the systemic circulation at the junction of the left subclavian vein and the left jugular vein, which is located immediately above the heart. High concentrations in the lymph are possible if the drug is administered in the fasted state, in contrast to the situation in the fed state where large quantities of exogenous food related lipids effectively dilute the drug within the lymph. Conversely, according to the present invention, it may be possible to formulate and deliver a medium to highly lipophilic drug to a subject in such a way, i.e. in the fed state, such that the concentrations of the highly lipophilic drug in the lymph is minimised. This may be particularly important in the case of lipophilic drugs which, if delivered to the heart in high concentration through the lymph, may have a deleterious effect on the heart, for example by prolonging the QTc interval as measured on an ECG. An example of such a drug may be the antimalarial drug, halofantrine. In view of this effect, the present invention provides a means for testing a medium to highly lipophilic drug to ensure that administration of the drug to a subject in the fasted state together with a small amount of an appropriate component, and the consequential high concentration of the drug delivered to the systemic circulation near the heart, does not cause prolongation of the QTc interval of an ECG. A further advantage of the present invention is that when a drug is formulated according to the invention, it is not necessary to specify that the formulation must be taken by the subject in the fed state, for example before, during or after a meal. This is because good bioavailability can be achieved in both the fed and fasted state when the drug is formulated according to the present invention. Of course, care would need to be taken during the trialing of any such formulation to ensure that the high concentration of the drug in the lymph (when administered in the fasted state) does not have a significant adverse effect on the heart of the subject. These advantages are in addition to the advantages inherent in a drug delivery system which substantially avoids passage of the drug to the liver via the portal blood.

As used herein the term “medium to highly lipophilic drug” refers to a drug which is capable of being absorbed into the lymph through the intestinal wall. As described above, in order to be preferentially transported into the lymph, as opposed to the portal blood, a drug should preferably have a log P (octanol/water)>4.7 and a triglyceride (TG) solubility, as measured, for example, by solubility in soybean oil or similar, in excess of 50 mg/mL. However drugs with a log P>2, may also be administered according to the present invention. Examples of medium to highly lipophilic drugs which may be suitable for formulation according to the present invention include the following: Analgesics and anti-inflammatory agents: aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac.

Anthelmintics: albendazole, bephenium hydroxynaphthoate, cambendazole, dichlorophen, ivermectin, mebendazole, oxamniquine, oxfendazole, oxantel embonate, praziquantel, pyrantel embonate, thiabendazole.

Anti-arrhythmic agents: amiodarone, disopyramide, flecainide acetate, quinidine sulphate.

Anti-bacterial agents: benethamine penicillin, cinoxacin, ciprofloxacin, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim.

Anti-coagulants: dicoumarol, dipyridamole, nicoumalone, phenindione.

Anti-depressants: amoxapine, maprotiline, mianserin, nortriptyline, trazodone, trimipramine maleate.

Anti-diabetics: acetohexamide, chlorpropamide, glibenclamide, gliclazide, glipizide, tolazamide, tolbutamide.

Anti-epileptics: beclamide, carbamazepine, clonazepam, ethotoin, methoin, methsuximide, methylphenobarbitone, oxcarbazepine, paramethadione, phenacemide, phenobarbitone, phenyloin, phensuximide, primidone, sulthiame, valproic acid.

Anti-fungal agents: amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine, terconazole, tioconazole, undecenoic acid.

Anti-gout agents: allopurinol, probenecid, sulphin-pyrazone.

Anti-hypertensive agents: amlodipine, benidipine, darodipine, dilitazem, diazoxide, felodipine, guanabenz acetate, isradipine, minoxidil, nicardipine, nifedipine, nimodipine, phenoxybenzamine, prazosin, reserpine, terazosin.

Anti-malarials: amodiaquine, chloroquine, chlorproguanil, halofantrine, mefloquine, proguanil, pyrimethamine, quinine sulphate.

Anti-migraine agents: dihydroergotamine mesylate, ergotamine tartrate, methysergide maleate, pizotifen maleate, sumatriptan succinate.

Anti-muscarinic agents: atropine, benzhexol, biperiden, ethopropazine, hyoscyamine, mepenzolate bromide, oxyphencylcimine, tropicamide.

Anti-neoplastic agents and Immunosuppressants: aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine, tamoxifen citrate, testolactone.

Anti-protazoal agents: benznidazole, clioquinol, decoquinate, diiodohydroxyquinoline, diloxanide furoate, dinitolmide, furzolidone, metronidazole, nimorazole, nitrofurazone, ornidazole, tinidazole.

Anti-thyroid agents: carbimazole, propylthiouracil.

Alixiolytic, sedatives, hypnotics and neuroleptics: alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, baloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone.

β-Blockers: acebutolol, alprenolol, atenolol, labetalol, metoprolol, nadolol, oxprenolol, pindolol, propranolol.

Cardiac Inotropic agents: amrinone, digitoxin, digoxin, enoximone, lanatoside C, medigoxin.

Corticosteroids: beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone.

Diuretics: acetazolamide, amiloride, bendrofluazide, bumetanide, chlorothiazide, chlorthalidone, ethacrynic acid, frusemide, metolazone, spironolactone, triamterene.

Anti-parkinsonian agents: bromocriptine mesylate, lysuride maleate.

Gastro-intestinal agents: bisacodyl, cimetidine, cisapride, diphenoxylate, domperidone, famotidine, loperamide, mesalazine, nizatidine, omeprazole, ondansetron, ranitidine, sulphasalazine.

Histamine H,-Receptor Antagonists: acrivastine, astemizole, cinnarizine, cyclizine, cyproheptadine, dimenhydrinate, flunarizine, loratadine, meclozine, oxatomide, terfenadine.

Lipid regulating agents: bezafibrate, clofibrate, fenofibrate, gemfibrozil, probucol.

Nitrates and other anti-anginal agents: amyl nitrate, glyceryl trinitrate, isosorbide dinitrate, isosorbide mononitrate, pentaerythritol tetranitrate.

Nutritional agents: betacarotene, vitamin A, vitamin B₂, vitamin D, vitamin E, vitamin K.

Opioid analgesics: codeine, dextropropyoxyphene, diamorphine, dihydrocodeine, meptazinol, methadone, morphine, nalbuphine, pentazocine.

Sex hormones: clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone.

Stimulants: amphetamine, dexamphetamine, dexfenfluramine, fenfluramine, mazindol.

The lipophilic drugs may be in free acid, free base or salt form, and mixtures of lipophilic drugs may be used where therapeutically effective.

The term “medium to highly lipophilic drug” also includes therapeutic compounds which have been modified, e.g. by attachment to a lipophilic moiety, to increase the lipophilicity of the drug to an extent that lymph transport is possible. Preferably the drug is a highly lipophilic drug, for example a drug having a log P>3.5, more preferably a log P>4.7.

The term “fasted state” as used herein refers to a state of the subject in which the only lipids, if any, present in the intestine of the subject, apart from any which may have been included in a formulation according to the invention, are endogenous lipids. A reference to the oral administration of a drug or formulation according to the invention to a subject “in the fasted state” is a reference to the oral administration into the digestive system of the subject such that during the uptake into the lymphatic system of a therapeutically effective amount of the drug, the subject is in the fasted state. This generally means that the subject has not taken a meal at least 3 to 4 hours prior to the administration and, depending on the rate of uptake and the efficacy of the drug, no food is taken from 1 to 6 hours after the meal.

The term “fed state” as used herein refers to any state of the subject other than a “fasted state” as described above.

The expression “production of endogenous lipid” as used herein refers to the biosynthesis within the intestinal absorptive cells of lipids, including mono, di or triglycerides and phospholipids, from bioprecursors, which bioprecursors could themselves be lipids or lipid conjugates, such as glycerides. For example the biosynthesis may involve the conversion of a lipid species unable to promote transport of the drug into the lymphatic transport system into a species which can. The term “production of endogenous lipid” may also refer to the translocation of lipid species into the enterocytes from elsewhere, such that the lipid species, or lipid metabolite thereof, is capable of promoting transport of the drug into the lymphatic transport system.

Unless a contrary intention appears the term “lipid” as used herein is understood to refer to saturated mono-unsaturated and polyunsaturated fatty acids and derivatives thereof. Suitable derivatives include ester derivatives, such as mono, di and triglycerides, as well as phospholipids or other glyceride esters.

The component capable of stimulating the production of endogenous lipid or otherwise enhancing or promoting lymphatic transport may be a lipid or combination of lipids.

The lipid may be a medium to long chain fatty acid of from C₈ to C₂₂, or a derivative thereof. Preferably the lipid is or includes an unsaturated long chain fatty acid of from C₁₄ to C₂₂, more preferably C₁₆ to C₁₈, or a mono, di or triglyceride thereof. The lipid is preferably a monounsaturated fatty acid or a derivative, such as a mono, di or triglyceride, or a phospholipid thereof. The term “long chain lipid” refers to long chain fatty acids, as well as derivatives of long chain fatty acids. Examples of lipids which may be suitable components for stimulating the production of endogenous lipid and which are known to be safe for human consumption include those described in U.S. Pat. No. 6,096,338, the entire contents of which is incorporated herein by reference.

Preferably the lipids are formulated with the highly lipophilic drug in the form of a naturally derived oil, such as soybean oil, olive oil, peanut oil, rapeseed oil, sunflower oil, coconut oil, corn oil, sunflowerseed oil, cotton seed oil, palm oil, arachis oil or a combination thereof.

The component capable of stimulating the production of endogenous lipid or otherwise enhancing or promoting lymphatic transport may be a lipid or lipids in combination with one or more pharmaceutically acceptable surfactants. Examples of surfactants which may be suitable include esters of mono or di-glycerides, such as the acetic, succinic, lactic, citric or tartaric esters, propyleneglycol mono or di-esters of fatty acids, poly glycerol esters of fatty acids, acid and ester ethoxylates of fatty acids, sorbitan esters of fatty acids, transesterification products of natural or hydrogenated vegetable oil triglycerides and polyalkylene polyol, alcohol ethoxylates, polyoxyethylene or polyoxypropylene co-polymers, phospholipids, polyoxyethylene sorbitan fatty acid derivatives, castor oil or hydrogenated castor oil ethoxylates, for example Cremophor EL™, anionic surfactants, such as sodium lauryl sulphate or sodium oleate, alkylphenol surfactants, as well as mixtures of such surfactants. In such combinations, the surfactant may act to assist uptake of the fatty acid from the intestinal lumen. In a preferred embodiment a generally hydrophilic surfactant with an HLB value>10, such as Cremophor EL™, is used, optionally in combination with a co-surfactant, which may be a hydrophobic surfactant with HLB values<10. When a surfactant is used in the absence of a medium to long chain lipid it is preferably a surfactant derived from a fatty acid or fatty acids.

The lipophilic drugs and stimulators of lipid production and/or enhancers or promoters of lymph transport, together with any conventional adjuvants, carriers or diluents, may be placed into the form of pharmaceutical compositions and unit dosages thereof, and in such form may be employed as solids, such as tablets, pills or filled capsules, or liquids such as solutions, suspensions, emulsions, elixirs, or capsules filled with the same, all for oral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. The actual dosage range will depend on the particular drug included in the formulation and the propensity of that drug to be absorbed from the intestinal cavity into the lymph. In some cases it will be necessary to allow for the fact that not all of the drug will pass through the intestinal wall and into the systemic circulation.

The amount of medium to long chain lipid or other stimulator of production of endogenous lipid, or enhancer or promoter of lymph transport, included in the formulation will be less than the amount required to transport the drug into the lymph in the absence of the production of further endogenous lipid or enhancement or promotion of lymph transport. The amount will generally be in the order of 0.05 to 4 g, more preferably 0.1 to 1 g, corresponding to an amount which could be readily incorporated into a single unit dosage form, such as a capsule, of the formulation.

For preparing pharmaceutical compositions according to the present invention, the pharmaceutically acceptable carriers or diluents can be either solid or liquid. Solid form preparations include powders, tablets, pills, capsules, sachets and dispersible granules. A solid carrier can be one or more substances which may also act as diluents, flavouring agents, solubilisers, lubricants, suspending agents, binders, preservatives, tablet disintegrating agents, or encapsulating material.

In powders, the carrier is a finely divided solid which is in a mixture with the finely divided active component, and a corresponding amount of stimulator of lipid production/enhancer or promoter of lymph transport.

In tablets, the components mixed with the carrier having the necessary binding capacity in suitable proportions and compacted in the shape and size desired.

The powders and tablets preferably contain from 5 or 10 to about 70% by weight of the active compound and from 2 to 60%, more preferably 5 to 40%, of the stimulator of lipid production (or enhancer or promoter of lymph transport), although the exact proportions will depend on the properties of the lipophilic drug and the ability of the stimulating component to stimulate lipid production in sufficient quantities to transport the drug into the lymph or the ability of the enhancer or promoter to enhance or promote lymph transport. Suitable carriers are magnesium carbonate, magnesium stearate, talc, sugar, lactose, pectin, dextrin, starch, gelatin, tragacanth, methylcellulose, sodium, carboxymethyl cellulose, a low melting wax, cocoa butter, and the like. The term “preparation” is intended to include the formulation of the defined compound with an encapsulating material as a carrier providing a capsule in which the active component, with or without carriers, is surrounded by a carrier, which is thus in association with it. Similarly, sachets and lozenges are included. Tablets, powders, capsules, pills, sachets, and lozenges can be used as solid forms suitable for oral administration. Liquid form preparations include solutions, suspensions, and emulsions, including microemulsions, which liquid form preparations may be encapsulated in a capsule or contained within vials or ampoules.

Also included are solid form preparations which are intended to be converted, shortly before use, to liquid form preparations for oral administration. Such liquid forms include suspensions and emulsions. These preparations may contain, in addition to the active component and the stimulator of lipid production, colourants, flavours, stabilizers, buffers, artificial and natural sweeteners, dispersants, thickeners, solubilising agents, and the like.

The pharmaceutical formulations of the present invention are preferably in unit dose form. In such form, the preparation is subdivided into unit doses containing appropriate quantities of drug and other component(s). The unit dose form will also contain appropriate quantities of diluents, flavouring agents, solubilisers, lubricants, suspending agents, binders, preservatives, tablet disintegrating agents, or encapsulating materials where necessary. The unit dose form can be package preparation, the package containing discrete quantities of preparation, such as packeted tablets, capsules, caplets and powders or liquids in vials or ampoules. Also the unit dosage form can be a capsule, tablet, caplet, cachet or lozenge itself, or it can be any number of these in packaged form.

The lipophilic drugs and stimulators of lipid production or enhancers or promoters of lymph transport are preferably formulated in a way which enhances uptake of the drug from the intestinal cavity. Such formulation types would be well known to a person skilled in the art and include lipid based emulsions and micro emulsions and self-emulsifying and self-microemulsifying formulations, in which the active component is highly dispersed in the presence of a surfactant, as well as lipid solutions and lipid suspensions. Self-emulsifying and self-microemulsifying capsule formulations are those which spontaneously form emulsions or microemulsions on rupture of the capsule and contact of the contents of the capsule with the gastric or intestinal fluids and which are commonly termed self-emulsifying drug delivery systems (SEDDS) or self-microemulsifying drug delivery systems (SMEDDS).

### DETAILED DESCRIPTION OF THE INVENTION

The invention will now be described with reference to the accompanying drawing and examples which illustrate an embodiment of the invention. However, it is to be understood that the particularity of the following description is not to supersede the generality of the proceeding description of the invention.

### 1. BRIEF DESCRIPTION OF THE DRAWING

Referring to the drawing:

FIG. 1 is a plot of cumulative lymphatic halofantrine transport versus time of various halofantrine formulations tested.

### 2. EXAMPLES

Chemicals, Reagents and Halofantrine (Hf) Formulation

Hf base was supplied by SmithKline Beecham Pharmaceuticals (Mysore, India), and PEG 6000 polyethylene glycol 6000) was from Ajax Chemicals (Australia). Acetylpromazine maleate (Delvet Pty Ltd, Australia), propofol (Schering-Plough, Australia), cephazolin (Sigma Pharmaceuticals, Australia), carprofen (Pfizer, Australia), Iohexol (Nycomed, Australia) and polydioxinone sutures (Ethicon, USA) were used as received. Normal Saline (0.9%) and Lactated Ringer's Solution (an isotonic solution containing 3.22 g/L sodium lactate, 6.0 g/L sodium chloride, 0.4 g/L potassium chloride and 0.27 g/L calcium chloride) were obtained from Baxter Healthcare (Australia). The oral formulation of Hf base (100 mg) was an amorphous PEG 6000 solid dispersion prepared by the fusion method.¹⁵ Briefly, crystalline Hf base was heated to produce the amorphous form prior to the addition of PEG 6000 for the fusion of drug and carrier at 70-80° C. The self-microemulsifying (SMEDDS) formulation is an isotropic mixture of long chain triglyceride (soybean oil—29% w/w), long chain mono- and di-glycerides (Maisine)—29%, Cremophor EL™—30%, ethanol—7% and halofantrine—5%. Soybean oil is a long chain triglyceride consisting 54% w/w linoleic acid (C18:2), 22% oleic acid (C18:1), 1% palmitic acid (C16), 9% linolenic acid (C18:3) and 4% stearic acid (C18:0) (certificate of analysis, Sigma Chemical Co., St Louis, Mo.). Maisine 35-1 is a blend of long chain mono-, di- and tri-glycerides (38% monoglyceride, 48% diglyceride, 13% triglyceride <1% fatty acid) consisting 57% w/w linoleic acid (C_{18:2}), 28% oleic acid (C_{18:1}), 11% palmitic acid (C₁₆), 2% stearic acid (C_{18:0}) and <1% linolenic acid (C_{18:3}), arachidic acid (C_{20:0}) and eicosenoic acid (C_{20:1}), (certificate of analysis, Gattefossé, Saint-Priest, France). The formulation is made by weighing the appropriate quantity of Hf free base into a glass tube, adding the appropriate quantities of excipients and stirring at 50° C. until all dissolved. The mixture is then cooled and the ethanol added with further stirring. The formulation is subsequently equilibrated at ambient temp for 48 h. This fill material is then filled into empty soft gelatin capsules using a syringe and needle such that a single capsule contains approximately 1 g of fill and 50 mg of Hf base. The capsules are sealed with molten gelatin.

Surgical and Related Procedures

All surgical and experimental procedures were performed in accordance with the guidelines and approval of the local Institutional Animal Experimentation Ethics Committee. Studies were conducted in male greyhound dogs (28-35 kg), and their health status was verified by a veterinarian prior to the study. Prior to surgery, each dog was fed a small lipid meal to facilitate identification of the thoracic duct. Surgical premedication included a subcutaneous injection of acetylpromazine maleate (0.5 mg/kg), after which anaesthesia was induced with an intravenous injection of propofol (3-6 mg/kg), and surgical anaesthesia maintained by delivery of halothane (1.5%) and oxygen. Each dog received intravenous infusion of Lactated Ringer's solution (300 mL/h) during surgery, and a post-operative injection of antibiotic (cephazolin, 20 mg/kg) and analgesic (carprofen, 4 mg/kg).

The ventral abdomen was firstly clipped and prepared for aseptic surgery. A 5 cm long ventral midline laparotomy was performed to allow a loop of jejunum to be exteriorised. A 133 mm 16 gauge cannula was then inserted into a jejunal vein to the level of the portal vein. The cannula was stabilised in both the portal and jejunal veins with a ligature of 3/0 silk to the jejunal vein and a minimal volume extension tube, filled with heparinised saline, was then attached and externalised through a separate stab incision in the left flank and stabilised with sutures. The laparotomy wound was closed in anatomical layers with 3/0 polydioxinone sutures. The position of the cannula in the portal vein was confirmed by a portal venogram following a 10 mL injection of a contrast dye (Iohexol), and cannula patency was maintained by small volume flushing with heparinised saline (1 U/mL).

The thoracic lymph duct was cannulated by an adaptation of a previous method (see for example N. E. Hoffman, The relationship between uptake in vitro of oleic acid and micellar solubilization. Biochim. Biophys. Acta 196 (1970) 193-203). The left ventral neck area and cranial thorax was clipped and prepared for aseptic surgery. A curved incision was made over the left external jugular vein and the vein was then dissected from the surrounding tissues to the axillary vein. At the junction of the internal jugular and brachiocephalic veins, the thoracic duct ampulla was identified by its whitish appearance This was then ligated at the point of entrance into the brachiocephalic vein and all tributaries converging to the thoracic duct were ligated to ensure the return of lymph through the main lymphatic canal. The thoracic duct was cannulated directly with PVC tubing (1.4 mm i.d.; 1.9 mm o.d., BioService, Australia) which was brought through the skin via a separate stab incision and stabilised in place with sutures and cyanoacrylate adhesive. The surgical wound was closed in layers with polydioxinone sutures. The externalised cannula either hung loosely from the neck (during recovery) or it was placed in a collection bottle which hung around the neck of the dog.

Following surgery, dogs recovered unrestrained in a closed run for 14-16 h during which they returned to normal ambulatory movement. A catheter was then inserted into the cephalic vein prior to drug administration to enable serial blood sampling during the study. At the conclusion of the study, each dog was sacrificed by an intravenous injection of pentobarbitone.

### Example 1

Experimental Procedures

For studies in the fasted state, dogs remained fasted throughout both the recovery and study periods. To obtain a post-prandial state, dogs were fed a standard can of commercial dog food (680 g) containing 5% crude fat (max) 30-45 min prior to drug administration. In a simple parallel design study, 100 mg Hf base prepared as an amorphous solid dispersion was orally administered with 50 mL of water to either fasted (n=3) or fed (n=4) dogs. In a separate leg 1×50 mg Hf in a SMEDDS formulation contained in a single soft gelatin capsule was also administered to fasted (n=4) dogs. To limit possible dehydration due to collection of thoracic lymph, 25 mL Normal Saline was administered hourly by IV bolus during the sampling period. Water was available ad libitum and the dogs were fed after collection of either the 10 or 12 h samples. Although the current study was limited to 24 h, study periods of 48 h could be readily achieved provided there was adequate fluid replacement.

Systemic and portal vein blood samples (2.5 mL) were obtained via indwelling cannulas at predose (−5 min), 0.5, 1, 1.5, 2, 2.5, 3, 3.5, 4, 5, 6, 8, 10 and 24 h following fasted and post-prandial oral administration, with an additional 12 h sample taken after post-prandial administration. Blood samples were collected into individual tubes containing dipotassium EDTA, plasma separated by centrifugation, and then stored at −20° C. prior to analysis.

After administration of Hf, lymph was continuously collected into 50 mL tubes containing 75 mg disodium EDTA (to prevent clot formation) for the 10-12 h post-dosing period. Individual lymph samples for each hourly collection period were combined and the mass of lymph collected per hour was determined gravimetrically. A 1 mL aliquot of lymph was then taken from each hourly sample, placed into individual 1.5 mL tubes and stored between 5-8° C. prior to analysis (within 24 h).

Analytical Procedures

Plasma concentrations of Hf and Hfm were determined using a validated HPLC assay (see for example Humberstone A. J., Currie G. J., Porter C. J. H, Scanlon M. J., Charman W. N., Simplified Liquid Chromatography Assay For the Quantitation of Halofantrine and Desbutylhalofantrine in Plasma and Identification of a Degradation Product of Desbuylhalofantrine Formed Under Alkaline Conditions, Journal of Pharmaceutical and Biomedical Analysis, 1995, 13, 265-272. Briefly, the limit of quantitation of the assay was 10 ng/mL for both Hf and Hfm and the assay was linear between 10 and 1000 ng/mL. The extraction efficiency was greater than 85%, and the intra- and inter-day coefficient of variation for Hf and Hfm were less than 15% across the concentration range (10 to 1000 ng/mL). Analysis of Hf in lymph involved dilution of 100 μL of lymph to 10 mL with acetonitrile and then vortexing for 1 min. The insoluble protein-based components were removed by centrifugation, and the supernatant was analysed by HPLC. Recovery of spiked Hf from blank lymph was greater than 95%. As all lymph draining from the thoracic duct was collected, the mass of drug transported was calculated by multiplying the lymph drug concentration by the corresponding mass of lymph from each collection period.

Lymph and plasma TG concentrations were measured using a Roche Cobas Mira clinical chemistry analyser (Basle, Switzerland) and a commercial enzyme-based colorimetric assay kit (Boehringer Mannheim, Germany). Post-prandial lymph lipoprotein fractions collected during the 1-2, 2-3 and 3-4 h periods were separated by ultracentrifugation in a Beckman SW 60 Ti rotor. Chylomicrons were firstly separated by layering lymph under a sodium chloride solution (d=1.0063 g/mL) followed by centrifugation at 44,100 rpm (15° C.) for 1.3 h. The bottom of the tube was then pierced with a needle to remove the remaining lymph, leaving the chylomicron fraction which had formed a white semi-solid plug at the top of the tube. The remaining lipoprotein fractions were separated using density gradient solutions and ultracentrifugation for 19 h at 58,000 rpm (15° C.). After fractionation, the amount of Hf associated with each lipoprotein fraction was determined by HPLC.

Data Analysis

The area under the plasma concentration-time profiles (AUC) was calculated using the linear trapezoidal method up to the last measured concentration. The peak plasma concentration (Cmax), and the time for the occurrence of the peak plasma concentration (Tmax), were noted directly from individual profiles. Differences in pharmacokinetic parameters between the treatment groups (fasted versus post-prandial administration) were analysed by Student's t-test at a significance level of α=0.05. A nonparametric Sign test at the 1% level was employed to determine if the difference between the portal and systemic plasma Hfm concentrations was greater than zero over the 10 h post-dosing period.

Results

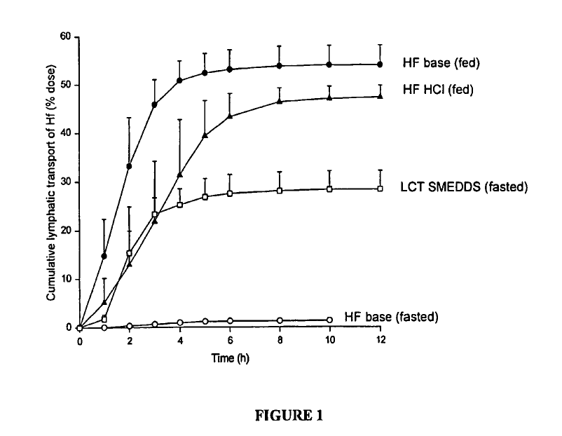

The results are shown in Table 1 while FIG. 1 shows the variation of lymph transport over time for the various formulations.

TABLE 1

| | Cum Hf | Cum TG | Transport into | |
|---|---|---|---|---|
| | transport in | transport in | syst circulation | Total BA |
| | lymph (% dose) | lymph (g) | (% dose) | (lymph & syst) |
| Hf base (fasted) | 1.3 +/− 0.7 | 0.7 +/− 0.5 | 30.2 +/− 18.7 | 31.5 +/− 8.3 |
| 100 mg (n = 3) | | | | |
| Hf base (fed) | 54.0 +/− 8.2 | 33.8 +/− 3.6 | 13.1 +/− 1.6 | 67.1 +/− 8.7 |
| 100 mg (n = 4) | | | | |
| Hf HCl (fed) | 47.3 +/− 4.3 | 32.5 +/− 6.7 | 19.0 +/− 8.1 | 66.2 +/− 12.4 |
| 107 mg(n = 3) | | | | |
| LCT SMEDDS | 28.3 +/− 7.5 | 3.7 +/− 2.2 | 29.4 +/− 8.6 | 57.7 +/− 7.3 |
| (fst) 50 mg Hf base | | | | |
| (n = 4) | | | | |
| IV (fed) 2 mg/kg | 1.5 +/− 0.2 | 29.0 +/− 4.4 | | |
| (n = 3) | | | | |

NB: Data are mean +/− sd, cumulative (cum) lymph transport for Hf and TG are over 12 h only for all except Hf base fst (0-10 h)

The total bioavailability (BA) of Hf in non-lymph cannulated beagles after administration of the LCT SMEDDS in the fasted state was found to be 67.3+/−21.0.

### Comparative Example 1

The formulation used in this comparative example was as follows:

| | |
|---|---|
| Halofantrine | 5% w/w |
| Captex 355 | 29% w/w |
| Capmul MCM | 29% w/w |
| Cremophor EL | 30% w/w |
| Ethanol | 7% w/w |

Captex 355 is a medium chain length triglyceride consisting 59% w/w caprylic acid (C8), 40% capric acid (C10), <1% lauric acid (C12) and <1% caproic acid (C6) (certificate of analysis, Abitec, Corporation, Janesville Wis.), Capmul MCM is a blend of medium chain mono-, di and tri-glycerides (58% monoglyceride, 36% diglyceride and 5% triglyceride by TLC) consisting 80% w/w caprylic acid (C8) 20% capric acid (C10) and 2% caproic acid (C6), average mol molecular weight 277 (certificate of analysis, Abitec, Corporation, Janesville Wis.).

The comparative example was conducted in exactly the same way as the example above except that two fasted greyhound dogs were each given 1×soft-gelatin capsule containing 1 g of the formula described above. The total mass Hf dosed to each animal was 50 mg. Lymph was collected and halofantrine and triglyceride in lymph assayed as described above.

Results

The results are shown in Table 2.

TABLE 2

| | Cumulative Hf transport in | Cumulative TG |
|---|---|---|
| | lymph (% dose) | transport in lymph (g) |
| Dog 1 | 6.0 | 0.90 |
| Dog 2 | 4.9 | 0.74 |
| Mean | 5.5 | 0.82 |

Data collected from 0-10 h

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

1. A method of delivering a medium to highly lipophilic drug to the systemic circulation through the lymphatic transport system comprising the oral administration to a subject in the fasted state of a formulation comprising said lipophilic drug and a component selected from medium to long chain lipids, pharmaceutically acceptable surfactants and combinations thereof, wherein said component is present in an amount sufficient to enhance or promote lymphatic transport of said lipophilic drug.

2. A method of delivering a medium to highly lipophilic drug to the systemic circulation through the lymphatic transport system comprising the oral administration to a subject in a fasted state of a formulation comprising said lipophilic drug and a component capable of stimulating sufficient production of endogenous lipid to allow transport of the drug into the lymphatic transport system.

3. A method of stimulating the production of endogenous lipid in a subject in a fasted state in sufficient quantity to enhance or promote transport of a medium to highly lipophilic drug into the lymphatic transport system for delivery to the systemic circulation, said method comprising orally administering to said subject in combination with said lipophilic drug an effective amount of a component selected from medium to long chain lipids, pharmaceutically acceptable surfactants and combinations thereof, whereby the amount of lipid transported to said lymphatic transport system following administration of said medium to long chain lipid drug is greater than the amount of lipid administered with said lipophilic drug.

4. A method of avoiding the requirement of administering a medium to highly lipophilic drug in the fed state, said method comprising formulating said lipophilic drug with a component selected from medium to long chain lipids, pharmaceutically acceptable surfactants and combinations thereof, such that oral administration of the formulation in the fed or fasted state allows delivery of the drug to the systemic circulation through the lymphatic transport system.

5. A method of avoiding the requirement of administering a medium to highly lipophilic drug in the fed state, said method comprising formulating said lipophilic drug with a component capable of stimulating the production of endogenous lipid, such that oral administration of the formulation in the fed or fasted state allows delivery of the lipophilic drug to the systemic circulation through the lymphatic transport system.

6. A method of delivering a medium to highly lipophilic drug in high concentration to the lymph comprising the oral administration to a subject in the fasted state of a formulation comprising said lipophilic drug and a component selected from medium to long chain lipids, pharmaceutically acceptable surfactants and combinations thereof, wherein said component is present in an amount sufficient to enhance or promote lymphatic transport of said lipophilic drug.

7. A method of delivering a medium to highly lipophilic drug in high concentration to the lymph comprising the oral administration to a subject in the fasted state of a formulation comprising said lipophilic drug and a component capable of stimulating sufficient endogenous lipid production to allow transport of the drug to the lymphatic transport system in high concentration.

8. A method of targeting delivery of a medium to highly lipophilic drug to the heart comprising the oral administration to a subject in the fasted state of a formulation comprising said lipophilic drug and a component selected from medium to long chain lipids, pharmaceutically acceptable surfactants and combinations thereof, wherein said lipid is present in an amount sufficient to enhance or promote lymphatic transport of said lipophilic drug.

9. A method of targeting delivery of a medium to highly lipophilic drug to the heart comprising the oral administration to a subject in a fasted state of a formulation comprising said drug and a component capable of stimulating sufficient production of endogenous lipid to allow transport of the drug into the lymphatic transport system.

10. A method of avoiding prolongation of the QT_{c} interval of an electrocardiogram (ECG) following oral administration of a medium to highly lipophilic drug, said method comprising the formulation of said lipophilic drug in the absence of medium to long chain lipids, or other component capable of stimulating the production of endogenous lipid, and the administration of said formulation in the fasted state.

11. A pharmaceutical formulation comprising a highly lipophilic drug and a component selected from medium to long chain lipids, pharmaceutically acceptable surfactants and combinations thereof, wherein said component is present in an amount sufficient to enhance or promote lymphatic transport of said lipophilic drug when administered in the fasted state.

12. A pharmaceutical formulation comprising a highly lipophilic drug and a component capable of stimulating sufficient production of endogenous lipid following administration to allow transport of the drug into the lymphatic transport system.

13. A method according to claim 1 wherein said component is selected from medium to long chain lipids and medium to long chain lipids combination with a pharmaceutically acceptable surfactant.

14. A method according to claim 1 wherein said component is selected from long chain lipids and long chain lipids in combination with a pharmaceutically acceptable surfactant.

15. A method according to claim 1 wherein the component is or includes a C₁₆ to C₁₈ fatty acid or derivative thereof, or a C₁₆ to C₁₈ fatty acid or derivative thereof in combination with a pharmaceutically acceptable surfactant.

16. A method according to claim 1 wherein the lipophilic drug has a log P>3.5 and a triglyceride solubility>50 mg/mL.

17. A method according to claim 16 wherein the lipophilic drug has a log P>4.7.

18. A method according to claim 15 wherein the fatty acid derivative is a mono, di or triglyceride.

19. A method according to claim 15 wherein the fatty acid is an unsaturated, monounsaturated or polyunsaturated fatty acid or a derivative thereof.

20. A method according to claim 1 wherein the pharmaceutically acceptable surfactant is a generally hydrophilic surfactant, optionally in combination with a co-surfactant.

21. A pharmaceutical formulation according to claim 11 in the form of a self emulsifying or self microemulsifying formulation.

22. A pharmaceutical formulation according to claim 21 wherein the formulation is an emulsion or microemulsion formulation.

23. A pharmaceutical formulation according to claim 11 wherein the lipid is present in an amount of from 0.05 to 4 g.

24. A pharmaceutical formulation according to claim 23 wherein the lipid is preset in an amount of from 0.1 to 1 g.
